(19) United States
(12) Patent Application Publication
(10) Pub. No.: US 2026/0175215 A1
(43) Pub. Date: Jun. 25, 2026

(54) MICROFLUIDIC DEVICE AND FABRICATION METHODS

(71) Applicants: Vi Nguyen, Phoenix, AZ (US); Jennifer Blain Christen, Chandler, AZ (US)

(72) Inventors: Vi Nguyen, Phoenix, AZ (US); Jennifer Blain Christen, Chandler, AZ (US)

(21) Appl. No.: 19/427,404

(22) Filed: Dec. 19, 2025

Related U.S. Application Data
(60) Provisional application No. 63/736,135, filed on Dec. 19, 2024.

(51) Int. Cl. B01L 3/00; C12Q 1/6844

(52) U.S. Cl. CPC B01L 3/502707; C12Q 1/6844; B01L 2200/027; B01L 2200/12; B01L 2300/048; B01L 2300/0681; B01L 2300/0887; B01L 2300/161

(57) ABSTRACT

A microfluidic device and fabrication method are provided. The microfluidic device includes a top cover with an inlet and a vent extending therethrough. Further, a layer is positioned beneath the top cover and includes a reaction chamber, a first channel, an inlet extension, and an outlet extension. The first channel is in fluid communication between the inlet and the reaction chamber, the inlet extension extends between the first channel and the reaction chamber, and the outlet extension extends between the reaction chamber and the vent. The reaction chamber, the inlet extension, and the outlet extension have an equal depth.

### RELATED APPLICATIONS

This application claims priority under 35 U.S.C. § 119 to U.S. Provisional Patent Application No. 63/736,135 filed on Dec. 19, 2024, the entire contents of which is incorporated herein by reference.

### BACKGROUND

Liquid assays, such as polymerase chain reaction (PCR) and loop-mediated isothermal amplification (LAMP), are difficult to implement in point-of-care (POC) microfluidic devices because bubbles often form. As microfluidics for liquid-based assays are prone to filling with bubbles, they often must be used in conjunction with some kind of metering system or actuator to regulate consistent flow rate. These additional necessary auxiliary systems complicate such tasks, requiring trained users, and are thus difficult to implement in low-resource settings. Furthermore, bubble formation eliminates the ability of such devices to be used for quantitative measurements, such as fluorescence-based assays. Rather, such devices may only be used for qualitative measurements, such as colorimetric assays.

Accordingly, it would be beneficial to have a microfluidic device design that can be used with liquid-based assays and human-actuated flow rates with minimal bubble formation. Such a design could thus be suitable for POC applications, including for both qualitative measurements and quantitative measurements.

### SUMMARY

Some embodiments provide a microfluidic device including a top surface and a layer positioned beneath the top surface. The top surface includes an inlet and a vent extending therethrough. The layer includes a reaction chamber, a first channel, an inlet extension, and an outlet extension. The first channel is in fluid communication between the inlet and the reaction chamber, the inlet extension extends between the first channel and the reaction chamber, and the outlet extension extends between the reaction chamber and the vent. The reaction chamber, the inlet extension, and the outlet extension have an equal depth and are co-planar.

Some embodiments provide a method of fabricating a microfluidic device. The method includes cutting, through a top cover with a cutting plotter, an inlet hole, a vent hole, and a first alignment hole. The method also includes cutting, through a layer with a cutting plotter, a second alignment hole, a first channel, a reaction chamber having a circular shape, an inlet extension extending from the reaction chamber, and an outlet extension extending from the reaction chamber. The reaction chamber, the inlet extension, and the outlet extension have an equal depth. The method further includes placing the top cover over an alignment fixture by aligning the first alignment hole of the top cover with a post of the alignment fixture, and placing the layer over the top cover by aligning the second alignment hole of the layer with the post of the alignment fixture to adhere together the top cover and the layer so that the inlet hole is in fluid communication with the first channel and the vent is in fluid communication with the outlet extension.

Some embodiments provide a method of performing a nucleic acid amplification assay. The method includes manually injecting a fluid sample into an inlet of a microfluidic device, the microfluidic device including a reaction chamber with an inlet extension and an outlet extension that are co-planar with the reaction chamber. The method also includes filling the reaction chamber with the fluid sample in a side-to-side manner through the inlet extension and the reaction chamber to the outlet extension. The method further includes heating the microfluidic device to perform nucleic acid amplification of a target in the fluid sample within the reaction chamber, and detecting the target using quantitative fluorescence measurement of the reaction chamber.

### DESCRIPTION OF THE DRAWINGS

FIG. 1 is an isometric view of a microfluidic device according to some embodiments.

FIG. 2A is depth view of an example reaction chamber, of the microfluidic device of FIG. 1, having a same-depth outlet (SDO) design.

FIG. 2B is depth view of another example reaction chamber, of the microfluidic device of FIG. 1, having a same-depth inlet outlet (SDIO) design.

FIG. 2C is depth view of yet another example reaction chamber, of the microfluidic device of FIG. 1, having a same-depth throughout (SDT) design.

FIG. 2D is a partial top view of the SDO reaction chamber of FIG. 2A.

FIG. 2E is a partial top view of the SDIO reaction chamber of FIG. 2B.

FIG. 2F is a partial top view of the SDT reaction chamber of FIG. 2C.

FIG. 3A is an exploded view of an example microfluidic device with an SDO reaction chamber design, according to some embodiments.

FIG. 3B is an exploded view of an example microfluidic device with an SDIO design, according to some embodiments.

FIG. 3C is an exploded view of an example microfluidic device with an SDT design, according to some embodiments.

FIG. 3D is an isometric view of an alignment fixture for use in fabricating a microfluidic device, according to some embodiments.

FIG. 3E is a top view of an example microfluidic device with an SDIO design, according to some embodiments.

FIG. 4 is a process flow of a method for fabricating a microfluidic device, according to some embodiments.

FIG. 5A is a graph illustrating instances of bubble formation with respect to flow rate when inputting a fluid sample into different microfluidic devices of some embodiments.

FIG. 5B is another graph illustrating the percentage of reaction chambers with bubbles formed when inputting a fluid sample into different microfluidic devices of some embodiments.

FIG. 5C is a top view of a fluid sample in a reaction chamber with bubbles formed in a well portion of the reaction chamber.

FIG. 5D is a top view of a fluid sample in a reaction chamber with bubbles formed outside a well portion of the reaction chamber.

FIG. 5E is a process flow of a fluid sample filling an example SDIO reaction chamber of some embodiments.

FIG. 5F is a process flow of a fluid sample filling a reaction chamber without same-depth extensions.

FIG. 6A is an exploded view of a microfluidic device made via xurographic methods, according to some embodiments, for testing nuclease contamination.

FIG. 6B is an isometric view of an alignment fixture for use in assembling the microfluidic device of FIG. 6A.

FIG. 6C is an exploded view of a microfluidic device made via laser-cut methods, according to some embodiments, for testing nuclease contamination.

FIG. 6D is an isometric view of an alignment fixture for use in assembling the microfluidic device of FIG. 6C.

FIG. 7A is a process flow of fluid sample collection for testing nuclease contamination with the microfluidic device of FIG. 6A.

FIG. 7B is a process flow of fluid sample collection for testing nuclease contamination and assay validation with the microfluidic device of FIG. 6C.

FIG. 8A is a graph of RNase contamination in a microfluidic device design shown in FIG. 6A, based on different assembly and treatment methods.

FIG. 8B is a graph of DNase contamination in a microfluidic device as shown in FIG. 6A, based on different assembly and treatment methods.

FIG. 8C is a graph of nuclease contamination in a microfluidic device as shown in FIG. 6C, based on different assembly and treatment methods.

FIG. 9A is a graph showing reaction chamber temperature over time of a microfluidic device as shown in FIG. 6C.

FIG. 9B is a graph comparing relative fluorescence of an empty reaction chamber of a laser-cut microfluidic device (shown in FIG. 6C) having covers made from different materials.

FIG. 9C is a graph comparing relative fluorescence of a reaction chamber of a laser-cut microfluidic device (shown in FIG. 6C), filled with positive and negative LAMP reaction mixes, and having covers made from different materials.

### DETAILED DESCRIPTION

Before any embodiments are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from previous embodiments. Thus, present embodiments are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of disclosed embodiments. Skilled artisans will recognize the examples provided herein have many useful alternatives and fall within the scope of embodiments of this disclosure.

Some embodiments provide microfluidic devices including one or more reaction chambers for liquid assays, such as loop-mediated isothermal amplification (LAMP) and polymerase chain reaction (PCR). The devices include reaction chamber designs that reduce bubble formation across a multitude of flow rates, thus solving the common issue of bubble formation in microfluidics without requiring auxiliary pumps or devices to control flow rate. The complete filling reproducibility of the reaction chamber designs allows the devices to be used with quantitative fluorescence detection, rather than being limited to qualitative colorimetric detection as with conventional bubble-prone designs. The reaction chamber designs are amenable to pressure-driven flow, allowing them to accommodate varying human-applied forces. Furthermore, the devices may be simple to fabricate, using xurography and/or laser cutting methods. Some embodiments also provide methods for reducing nuclease contamination of the devices through material handling and device treatment. Accordingly, the devices and methods herein may be optimal for use in point-of-care (POC) settings, which may have low resources and untrained personnel. The reaction chamber designs may be integrated into any microfluidic chip layout to accommodate existing or new device configurations, allowing the bubble-reducing technology to be broadly applied across different diagnostic platforms and applications. The devices may also be configured for multiplexed detection, where multiple reaction chambers are used to simultaneously detect different targets.

For example, the World Health Organization (WHO) has suggested that ideal POC diagnostic devices for developing countries should be affordable, sensitive, specific, user-friendly, rapid and robust, equipment-free, and deliverable to end-users (considered the “WHO ASSURED” criteria). There is a need for such portable POC systems designed for resource-limited settings, some of which may not have stable electricity. Additionally, the COVID-19 pandemic spawned huge interest in reverse-transcription LAMP (RT-LAMP) because of its potential for low-cost and rapid POC deployment. Simplicity, reliability, manufacturability, scalability, profitability, and end user usability are also considerations when designing such devices. The microfluidic devices of some embodiments herein can meet the WHO ASSURED criteria, and may further be simple to use, scalable, and reliable to enable commercial success.

More generally, isothermal nucleic acid amplification, such as LAMP, can offer a competitive advantage over the commonly used PCR because of the elimination of multiple temperature cycles. PCR is considered the gold standard in molecular diagnostics but is difficult to implement in POC settings. In order to achieve the temperature cycles needed for PCR, a sample can be moved through microfluidics where position dictates a localized temperature, or the sample can remain in one place for active heating and cooling. Serpentine microfluidics with continuous flow require a pump to move the sample through different heating areas, and the fixed microfluidic design does not allow for easy assay or protocol changes. Active heating and cooling methods such as Peltier temperature control require high power and costs that are not amenable to POC devices, especially those designed for low-resource settings. In contrast, isothermal nucleic acid amplification offers simpler and cheaper design implications for POC applications. The process uses four to six primers and strand displacement to amplify template nucleic acids at a single temperature. There are several options to detect the results of LAMP assays, including turbidimetric, colorimetric, fluorescence, and electrochemical detection.

Thus, LAMP can provide a good option for POC applications and microfluidics using low sample volumes. Microfluidics have been developed for LAMP assays for gene detection, as well as for RT-LAMP to detect RNA viruses, as noted above. Microfluidic device (chip) architecture ranges from disk-based chips fabricated with pressure sensitive adhesive and poly(methyl methacrylate) (PMMA) for detecting single genes, to multi-channel chips made by the soft lithography method with polydimethylsiloxane (PDMS) for multiple gene detection. Disposable RT-LAMP microfluidic chips have been reported, and some systems are also constructed with PDMS. Paper-based microfluidic chips for LAMP have also been developed, but these can have nonspecific binding and autofluorescence of the paper used. A popular method of making microfluidics is to use photolithography to pattern photoresist, which is then used as a mold for PDMS; however, this method requires an expensive cleanroom environment, and any design changes require a repeat of the entire lengthy process.

On the other hand, a technique called xurography, or razor writing, emerged as a rapid and inexpensive alternative that uses a cutting plotter to make structures on different types of films, which are then layered to create 3D channels. Xurography allows rapid design, fabrication, and testing of prototype microfluidics that can be scaled to production designs utilizing plastic injection molding. Similarly, laser plotting can be used to cut thicker materials which can also be assembled in layers. Laminate microfluidics involves the stacking of individually cut layers to form reservoirs and channels. Common materials used in laminate microfluidics include adhesive tapes, polymers, and glass, but this technique is compatible with a wide range of materials which can be chosen based on specific needs, such as low autofluorescence or optical clarity.

Channels within such chips made from adhesive tapes have shown 98% recovery of DNA that pass through, which indicates biocompatibility. Furthermore, various types of adhesive tapes and thicknesses have been tested in microfluidic chips to demonstrate high-quality bonding strength for long-term storage up to two months. Studies have been reported for accuracy of xurographic techniques, as well as inhibition of common microfluidic materials on PCR. Specifically, researchers have used PCR adhesive sealing films for building microfluidic chips to show that the off-the-shelf material is a low-cost option that is optically clear, can securely bond when stacked in layers, and can function at broad temperature ranges. Manually pressed PCR adhesive films can be removed for sample collection or rinseable and reusable chips, and this sealing method has been shown to work for on-chip PCR with cycling temperatures.

Accordingly, embodiments herein describe the designs, fabrication of, and decontamination methods for laminated xurography-based microfluidic devices for nucleic acid amplification. Furthermore, it should be noted that the designs and decontamination methods described herein may also be applied to microfluidic devices fabricated through laser cutting or other methods. The xurographic and laser cutting fabrication methods described herein may also be scaled to high-volume production. For example, roll-to-roll manufacturing processes may be used to produce the laminated microfluidic devices at commercial scale with little change to the design or materials described herein.

Looking now to FIG. 1, an example microfluidic device 10, according to some embodiments, is illustrated. The device 10, or chip, can include a device inlet 12 and one or more vents 14 extending through a top surface, such as a top cover 16. While ten vents 14 are illustrated in the embodiment of FIG. 1, more or fewer vents 14 may be implemented in some embodiments.

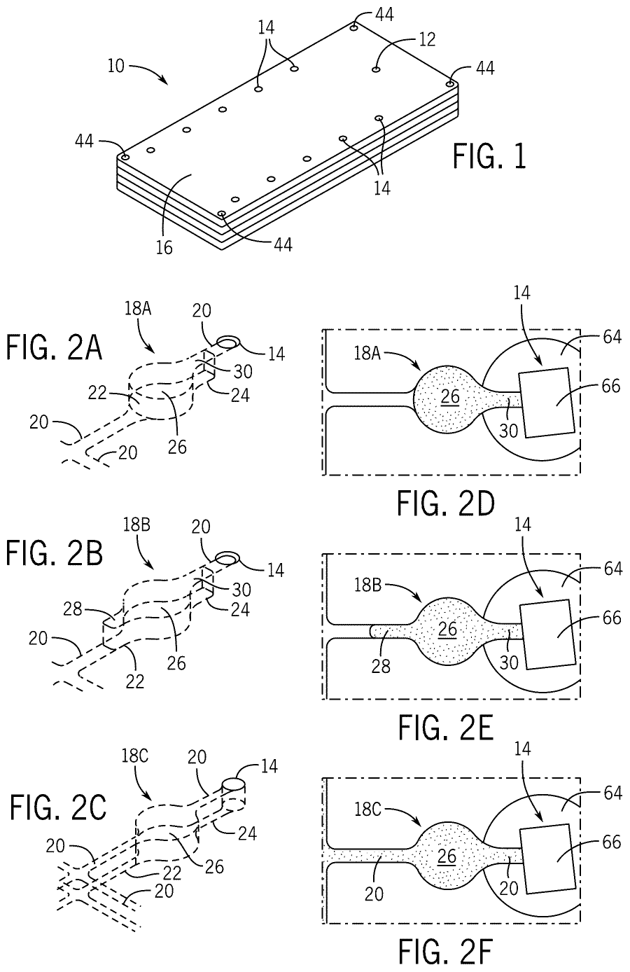

Each vent 14 of the microfluidic device 10 can be associated with a respective reaction chamber 18, connected to the inlet 12 via internal channels 20, as shown in FIGS. 2A-2B. The reaction chambers are denoted throughout generally as numeral 18, with specific designs shown in FIGS. 2A-2C as numerals 18A-18C, respectively. In use, a fluid sample can be injected into the device inlet 12, manually via a syringe or automatically via a pump or other device, and the fluid sample can travel from the device inlet 12, through the channels 20, to fill respective reaction chambers 18. The reaction chambers 18 are configured to accommodate pressure-driven flow, enabling reliable filling across a range of manually applied pressures. These reaction chamber designs can be integrated into any chip layout, supporting both existing and new microfluidic configurations.

Accordingly, FIGS. 2A-2C illustrate example reaction chambers 18A-18C, respectively, according to some embodiments. Each reaction chamber 18 is built upon a circular well design, as circular geometry fills better than rectangular geometry. Each reaction chamber 18 can include a chamber inlet 22 adjacent to and in fluid communication with a respective channel 20, a chamber outlet 24 adjacent to and in fluid communication with a respective vent 14 via a channel 20, and a well 26 between the chamber inlet 22 and the chamber outlet 24.

Looking to FIG. 2A, a reaction chamber 18A can include an outlet extension 30 between the well 26 and the chamber outlet 24 that is co-planar with and at the same depth as the well 26, while the channels 20 are a shorter depth than the reaction chamber 18A. As such, this reaction chamber design of FIG. 2A may be considered a same-depth outlet (SDO) design. FIG. 2B illustrates a reaction chamber 18B that includes an outlet extension 30 between the well 26 and the chamber outlet 24, and an inlet extension 28 between the well 26 and the chamber inlet 22, where the outlet extension 30 and the inlet extension 28 are co-planar with and at the same depth as the well 26, while the channels 20 are a shorter depth than the reaction chamber 18B. As such, this reaction chamber design of FIG. 2B may be considered a same-depth inlet outlet (SDIO) design. FIG. 2C illustrates a reaction chamber 18C that is co-planar with and at the same depth as all channels 20 leading to the reaction chamber 18C and to the vent 14. In one example, the channels 20 leading to and away from the reaction chamber 18C may be considered inlet and outlet extensions, respectively. As such, this reaction chamber design of FIG. 2C may be considered a same-depth throughout (SDT) design.

FIGS. 2D-2F illustrate microscopic images of each assembled reaction chamber design filled with dyed water to show a filled reaction chamber 18, with FIG. 2D illustrating a filled SDO reaction chamber 18A, FIG. 2E illustrating a filled SDIO reaction chamber 18B, and FIG. 2F illustrating a filled SDT reaction chamber 18C. For all shapes, a channel 20 leads into the reaction chamber 18, and a channel 20 leads out of the reaction chamber 18 to a top surface with a vent 14. Also shown in FIGS. 2D-2F are annular rings 64 surrounding each vent 14 and membrane filters 66 covering each vent 14, as further described below.

In one application, the channels 20 may be about 1 millimeter (mm) wide. The diameter of the circular wells 26 may be about 4 mm, and the extensions 28, 30 leading into and out of the wells 26 may be about 2.25 mm long. For the SDO reaction chamber 18A and the SDIO reaction chamber 18B, the total volume of the channels 20 may be about 22.5 microliters (μL). The SDO reaction chamber 18A may have a volume of about 24.5 μL and the SDIO reaction chamber 18B may have a volume of about 29 μL. For example, a typical LAMP reaction, such as the WarmStart® LAMP Kit from New England BioLabs, has a working reaction volume of 25 μL. Similarly, a standard 384-well PCR plate has a working volume of 25 μL. As such, for custom POC devices, designing close to the 25-μL volume (e.g., approximately 24.5-29 μL) can allow for benchmarking and comparison with existing products. However, as POC devices continue to be optimized for detection in the future, it is possible the volume may be scaled down. Furthermore, a volume of a vent 14 may be about 1.4 μL for the SDO reaction chamber 18A and about 1.6 μL for the SDIO reaction chamber 18B. As such, the total volume of a chip 10 with the SDO reaction chamber design may be about 281.5 μL and about 329.5 μL for the SDIO reaction chamber design. For a chip 10 with the SDT reaction chamber design, the total volume may be about 382 μL because all connecting channels 20 and reaction chambers 18C have the same depth.

While the specific embodiments described have reaction chamber volumes of approximately 24.5 μL to 29 μL, the design principles may be applied to reaction chambers having volumes ranging from 5 μL to 100 μL, or from 10 μL to 50 μL in some embodiments, depending on the specific assay requirements. That is, the dimensions provided above represent one working embodiment, but the design principles may be applied across a range of dimensions. For example, in some embodiments, the channels 20 may have a width between 0.5 mm and 2 mm, and the extensions 28, 30 may have a length of at least 2 mm, at least 2.25 mm, or between 2 mm and 5 mm. The circular well 26 may have a diameter between 2 mm and 10 mm, or between 3 mm and 6 mm in some embodiments. The depth of the channels 20 relative to the reaction chamber 18 may also vary. In some embodiments, the channels 20 may have a depth that is 5% to 100% of the reaction chamber depth, or may be up to 95% shorter than the overall reaction chamber 18 height. These ranges allow for customization based on desired reaction volumes, available fabrication methods, and specific assay requirements. Furthermore, the microfluidic devices described herein may accommodate flow rates between 100 μL/min and 1000 μL/min, or between 200 μL/min and 800 μL/min in some embodiments.

Additionally, the SDIO and SDO designs may provide advantages for multiplexed applications, where multiple reaction chambers 18 are used to detect different targets simultaneously. By limiting the same-depth extensions 28, 30 to only the inlet and outlet regions of the reaction chambers 18, while keeping the connecting channels 20 at a shallower depth, the overall volume of the device 10 may be reduced compared to the SDT design. This volume reduction may lower reagent costs and reduce the amount of sample needed. Furthermore, the reduced channel volumes may provide better fluidic separation between different reaction chambers 18, which may be beneficial for multiplexing applications where different primer targets are analyzed in separate chambers. The fluidic isolation may minimize cross-contamination between chambers 18 and allow each reaction chamber 18 to function independently.

The reaction chamber designs described herein may be particularly advantageous for point-of-care applications because they accommodate varying human-applied forces that create dynamic flow rates. Unlike conventional microfluidic devices that require auxiliary mechanical or pneumatic actuators to regulate consistent flow rates, the devices of some embodiments can be operated with simple manual actuation, such as hand-operated syringes, while maintaining reliable bubble-free filling. In particular, the co-planar configuration of the inlet extension 28, the well 26, and the outlet extension 30 in the SDIO and SDO designs enables side-to-side filling (e.g., a horizontal fluid progression across the chambers) rather than bottom-to-top filling, which significantly reduces the formation of air bubbles that can interfere with assay detection. This design is amenable to pressure-driven flow, accommodating dynamic human forces and varying flow rates.

The reaction chamber designs described herein may be implemented using various fabrication methods and materials, with the choice depending on available resources, desired production scale, and specific application requirements. The modular nature of the layered construction allows for flexibility in material selection while maintaining the functional advantages of the same-depth extensions. In some embodiments, the devices may be fabricated using readily available materials and equipment, making them accessible for rapid prototyping and small-scale production. The following sections describe specific fabrication approaches that have been validated for creating microfluidic devices with the reaction chamber designs described above, including both xurographic methods using cutting plotters and laser cutting methods for thicker materials. These fabrication techniques may enable translation from laboratory prototypes to production-scale devices while preserving the bubble-reducing features of the reaction chamber designs.

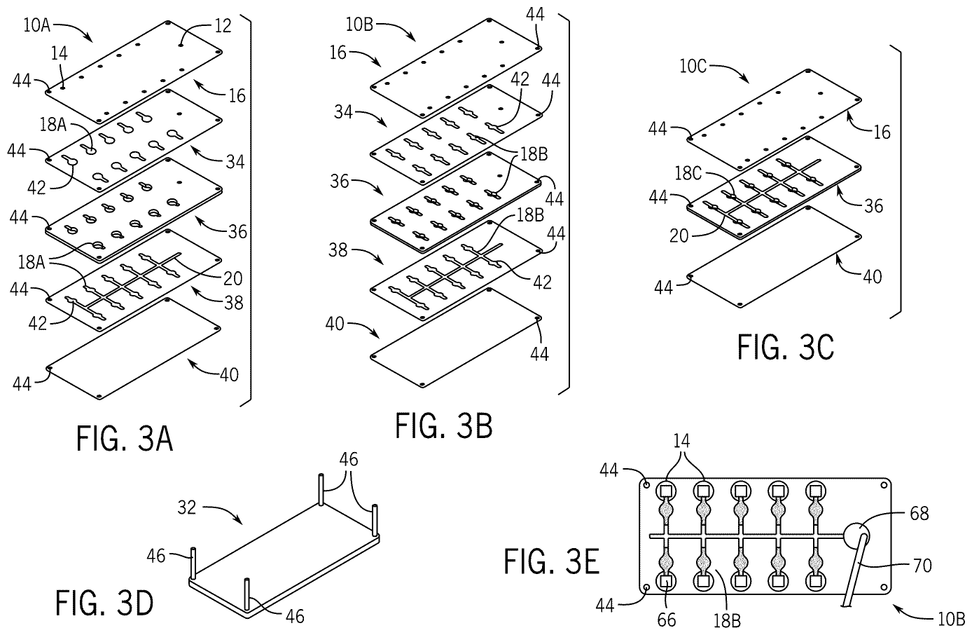

Turning now to FIGS. 3A-3E, various views of devices 10 including each reactor chamber design are illustrated. In particular, FIG. 3A illustrates an exploded view of an example device 10A including an SDO reaction chamber design, FIG. 3B illustrates an exploded view of an example device 10B illustrating an SDIO reaction chamber design, and FIG. 3C illustrates an exploded view of an example device 10C illustrating an SDT reaction chamber design. Furthermore, FIG. 3D illustrates an isometric view of an alignment fixture 32 that may be used during device fabrication, as further described below, and FIG. 3E illustrates a top view of an assembled device 10B from FIG. 3B.

Referring to FIGS. 3A and 3B, the device 10A including the SDO reaction chamber design and the device 10B including the SDIO reaction chamber design can include the same components in some embodiments. For example, each device 10A, 10B can include a top cover 16, one or more layers disposed beneath the top cover 16, such as an upper layer 34, a middle layer 36, a lower layer 38, and a bottom cover 40. In some embodiments, the upper layer 34 and the lower layer 38 can be double-sided pressure sensitive adhesive (PSA) tape, which may include cutouts 42 that form the channels 20, alignment holes 44 at each corner for alignment, and in the upper layer 34, a hole corresponding to the device inlet 12. More specifically, the lower layer 38 can be cut with channels 20 that lead from the inlet hole to the reaction chambers 18A/18B, and the upper layer 34 can be cut with channels 20 that lead from the reaction chambers 18A/18B to the vents 14. In some embodiments, the upper layer 34 and the lower layer 38 are made from 81 μm double-sided PSA tape (90445Q, Adhesives Research®).

In some embodiments, the middle layer 36 can be made of acrylic, such as a 1.5 mm thick clear cast acrylic (McMaster-Carr), which can be laser cut to define the reaction chambers 18A/18B, a hole corresponding to the device inlet 12, and holes 44 at each corner for alignment. Notably, the designs between the devices 10A, 10B differ in the cuts made to the upper layer 34 and the middle layer 36 to account for the different-shaped reaction chambers 18A, 18B. Additionally, covering the device 10A/10B on both sides are the top cover 16 and the bottom cover 40. In some embodiments, the top cover 16 and the bottom cover 40 can be made from 75 μm Melinex 454 polyethylene terephthalate film (PET, also known as polyester) (Tekra). The bottom cover 40 may only include corner holes 44 for alignment, while the top cover 16 can include alignment holes 44, holes for the vents 14, as well as a hole for the device inlet 12. In one embodiment, when assembled, the devices 10A, 10B can be about 80 mm long, about 36 mm wide, and about 1.9 mm high.

Referring to FIG. 3C, the device 10C including the SDT reaction chamber design can include a top cover 16, a middle layer 36, and a bottom cover 40. That is, the device 10C does not require the upper layer 34 and the lower layer 38 of double-sided PSA tape to create the channels 20, as the channels 20 may instead be cut into the middle layer 36. Accordingly, the middle layer 36 can be made of acrylic, such as a 1.5 mm thick clear cast acrylic (McMaster-Carr), which can be laser cut to define the channels 20, the reaction chambers 18C, a hole corresponding to the device inlet 12, and holes 44 at each corner for alignment. Additionally, covering the device 10C on both sides are the top cover 16 and the bottom cover 40. In some embodiments, the top cover 16 and the bottom cover 40 can be made from single-sided PSA tape (MTC Bio) used as a sealing film. The bottom cover 40 may only include corner holes 44 for alignment, while the top cover 16 can include holes for the vents 14 as well as a hole for the device inlet 12. In one embodiment, when assembled, the device 10C can be about 80 mm long, about 36 mm wide, and about 1.7 mm high.

Referring still to FIGS. 3A-3C, in some embodiments, the upper layer 34, the lower layer 38, the top cover 16, and/or the bottom cover 40 can be cut with a device such as, but not limited to, a Cricut® Explore (Cricut®, Inc.). The middle layer 36 can be cut with a device such as, but not limited to, a CO2 laser (VLS4.75, Universal Laser Systems). In some embodiments, the devices 10 can be assembled using an alignment fixture 32, as shown in FIG. 3D. For example, in some embodiments, the alignment fixture 32 can be about the same width and length as the device 10. Furthermore, in one embodiment, the alignment fixture 32 can be made using laser cut cast acrylic, with gauge 18 zinc nails 46 about 19 mm long (Everbilt) extending upward from corners thereof. The corner holes 44 of the layers 34-38 and covers 16, 40 of the devices 10 can be aligned with the nails 46 during device assembly.

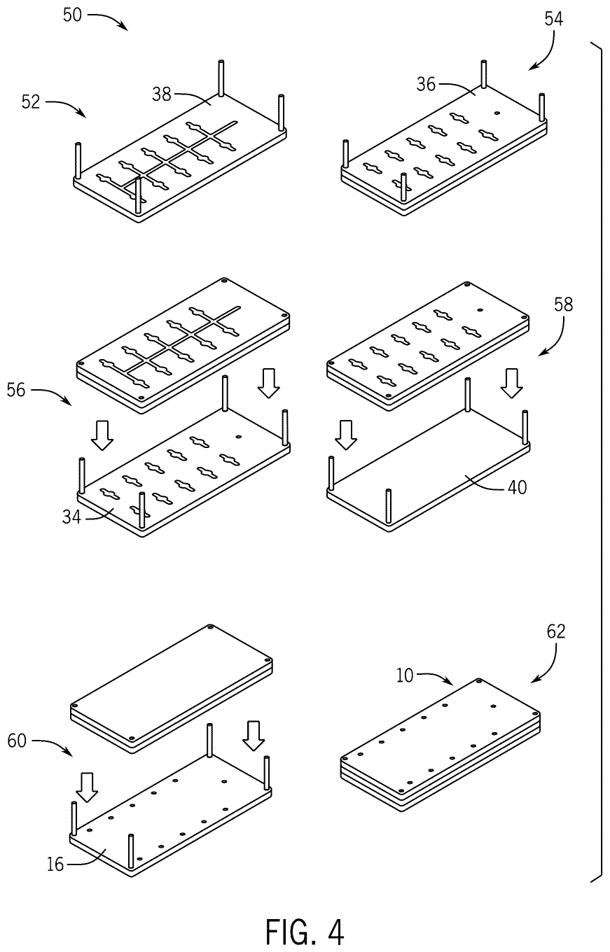

More specifically, FIG. 4 illustrates an example device assembly process flow 50, according to some embodiments, for fabricating the SDIO device 10B of FIG. 3B (and, though not shown, the SDO device 10A of FIG. 3A). As shown in FIG. 4, at step 52, the lower layer 38 can be placed on the alignment fixture 32 (e.g., by aligning the corner holes 44 of the lower layer 38 over the nails 46 of the alignment fixture 32. At step 54, the middle layer 36 can be placed onto the alignment fixture 32 on top of the lower layer 38, adhering the layers together, and the now two-layer assembly can be removed from the alignment fixture 32 and flipped over. At step 56, the upper layer 34 can be placed on the alignment fixture 32, and the two-layer assembly from step 54 can be placed on top of the upper layer 32, adhering the components together. The now three-layer assembly can be removed from the alignment fixture 32 and flipped over. At step 58, the bottom cover 40 can be placed on the alignment fixture 32 and the three-layer assembly from step 56 can be placed on top of the bottom cover 40, adhering the components together. The now four-layer assembly can be removed from the alignment fixture and flipped over. At step 60, the top cover 16 can be placed on the alignment fixture 32, and the four-layer assembly can be placed on top of the top cover 16, adhering the components together. At step 62, the assembled device 10 can be removed from the alignment fixture 32. Furthermore, in some embodiments, the assembled device 10 can be placed in a press to ensure adhesion. For example, the assembled device 10 can be placed between aluminum plates with rubber backing and pressed with a 500-lb capacity arbor press (McMaster-Carr) to ensure adhesion.

In some embodiments, once the device 10 is assembled at step 62, annular rings 64 can be cut and one ring placed over each vent hole 14, as best shown in FIGS. 2D-2F. For example, the annular rings 64 can be made of an adhesive material, such as double-sided acrylic adhesive tape (300LSE, 3M). To prevent fluid leakage out the vents 14, hydrophobic membrane filters 66 (e.g., 0.22 μm PTFE membrane filters (SF14684, Tisch Scientific)) can be cut and placed over the vents 14 with the annular ring adhesives 64. Additionally, in some embodiments, a connector 68 can be placed over the device inlet 12, as best shown in FIG. 3E. For example, in one embodiment, a press fit tubing connector 68 (460003, Grace Bio-Labs) can be placed over the device inlet 12 to enable connection to a syringe 70 or syringe pump. An example of a completed device 10B, including the filters 66 and the connector 68, is illustrated in FIG. 3E.

Referring back to the SDT device 10C of FIG. 3C, the device 10C may be assembled in a similar manner as that described above with reference to the process 50 of FIG. 4 for assembling the SDO and SDIO devices 10A/10B. For example, the bottom cover 40 can be placed on the alignment fixture 32, and the middle layer 36 can be placed onto the alignment fixture 32 on top of the bottom cover 40, adhering the layers together. The now two-layer assembly can be removed from the alignment fixture 32 and flipped over. Top cover 16 can then be placed on the alignment fixture 32, and the two-layer assembly can be placed on top of the top cover 16, adhering the components together. The assembled device 10C can be removed from the alignment fixture 32 and, in some embodiments, can be placed in a press to ensure adhesion. Annular ring adhesives 64 and hydrophobic filters 66 can be placed over the vent holes 14, and a press fit tubing connector 68 can be placed over the device inlet 12 for a syringe pump.

Generally, the materials used in the microfluidic devices described herein may be selected to withstand the temperatures required for nucleic acid amplification assays. By way of example, LAMP assays typically operate at temperatures between 60° C. and 65° C., while PCR requires cycling between approximately 50° C. and 95° C. The PET, PSA tape, and acrylic materials described herein are stable at these temperatures.

While the above description focuses on xurographic and laser cutting fabrication methods, the microfluidic device designs of some embodiments are compatible with various other fabrication techniques. In some embodiments, devices 10 may be fabricated via injection molding, which may be advantageous for high-volume commercial production and can reduce per-unit costs at scale. In injection molding embodiments, the device layers described above may be combined into a single monolithic structure, with the reaction chambers 18, channels 20, and extensions 28, 30 formed as cavities within the molded body. A top cover may then be bonded or sealed to the molded body. In other embodiments, devices 10 may be fabricated via three-dimensional (3D) printing using stereolithography, fused deposition modeling, or other additive manufacturing techniques. 3D printing may enable rapid prototyping and design iteration. The same-depth inlet and outlet extensions 28, 30 that reduce bubble formation may be created in 3D printed devices by ensuring the inlet extension 28, reaction chamber 18, and outlet extension 30 are formed at the same height within the printed structure. Accordingly, in some applications, injection molding or 3D printing fabrication methods may reduce assembly time.

In view of the above description of the microfluidic devices 10 and their methods of fabrication, according to some embodiments, the following paragraphs describe fluid filling capacity and bubble formation, nuclease contamination, and assay validation associated with such device designs.

### Fluid Filing Capacity

As noted above, the microfluidic devices 10 of some embodiments can be used for liquid assays with minimal bubble formation. A set of experiments was performed to determine the reaction chamber shape and flow rate least prone to filling with bubbles. As described above with reference to FIGS. 2A-2C, three reaction chamber designs were tested: the SDO design (FIG. 2A), the SDIO design (FIG. 2B), and the SDT design (FIG. 2C).

In typical nucleic acid isolation kits using spin columns, a biological sample is mixed with a binding buffer and passed through a silica matrix. Alternatively, magnetic beads can be used to isolate nucleic acids. The experiments focused on an implementation of a silica matrix because of the perceived simplicity of integrating it onto a microfluidic device relative to magnetic beads. It may be assumed that there is some residual buffer remaining in the silica matrix after the biological sample mix passes through and that the nucleic acids will be eluted with Tris-EDTA (TE) buffer. A fluid mixture containing 25% binding buffer was mixed to simulate the fluid that would be flowing through the reaction chambers 18. The binding buffer was made similar to some commercial buffers by using 1 M GuHCI (Sigma-Aldrich, St. Louis, MO), 10 mM Tris HCl (Sigma-Aldrich, St. Louis, MO), and 1 mM EDTA (G-Biosciences, St. Louis, MO). The ratio of the fluid mixed was 250 μL binding buffer, 745 μL TE buffer (pH 8.0, VWR Life Science, Solon, OH), and 5 μL blue food color (McCormick Culinary). This volume of simulated fluid was scaled up as needed. A Chemyx Fusion 101 syringe pump was used with gauge 23 dispensing needles, Tygon® microbore tubing (Cole-Parmer), and 3 mL luer lock syringes (BD).

In the experiments, each microfluidic device 10 (including the SDO, SDIO, and SDT designs from FIGS. 2A-4) was tested with three different flow rates and three repetitions each, for a total of 27 chips. The flow rates used were 250, 500, and 750 μL/min, to simulate low, medium, and high flow rates. The devices 10 were allowed to fill until fluid reached all vents 14, and each reaction chamber 18 was imaged using a Nikon SMZ1270 stereo microscope with Nikon Digital Sight 1000 camera. ImageJ was used to analyze any bubbles that formed in the reaction chambers to quantify their size. Outlines of bubbles were traced with the freehand selection tool, and their area was measured.

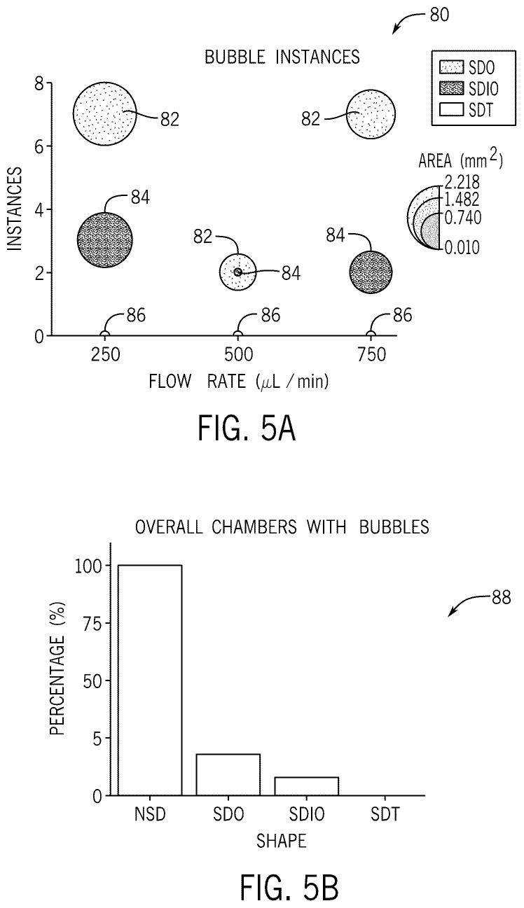

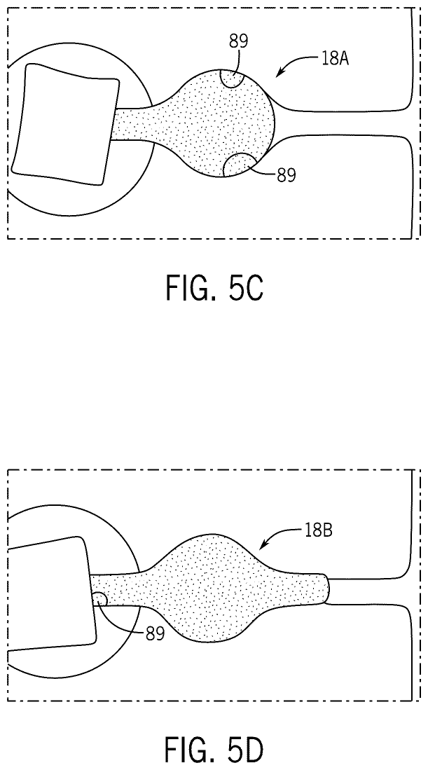

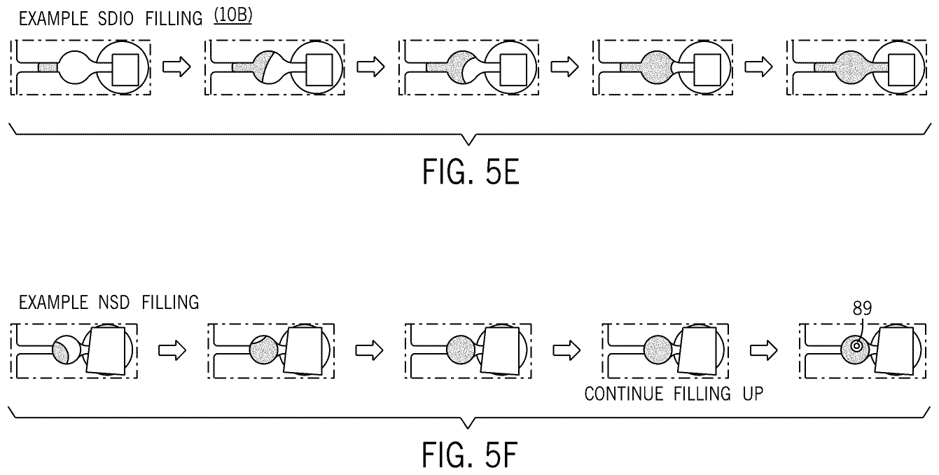

The experimental results demonstrated that adding same-depth extensions to circular wells 26 significantly reduced bubble formation across all tested devices 10A, 10B, 10C. The number of bubble instances per shape and flow rate is shown in the graph 80 of FIG. 5A. There were three chips 10 for each flow rate per shape, so each combination had thirty reaction chamber repetitions. In the graph 80 of FIG. 5A, the size of the circles is proportional to the bubble area. The individual bubble areas ranged from 0.008 millimeters squared (mm²) for SDIO reaction chamber designs at 500 μL/min, to 5.39 mm² for SDO reaction chamber designs at 250 μL/min. For the devices 10A containing the SDO reaction chamber design, represented by bubbles 82, the average bubble areas at 250 μL/min, 500 μL/min, and 750 μL/min were respectively 2.21 mm², 0.76 mm², and 1.32 mm². For the devices 10B containing the SDIO reaction chamber design, represented by bubbles 84, the average bubble areas at 250 μL/min, 500 μL/min, and 750 μL/min were respectively 1.71 mm², 0.019 mm², and 1.00 mm². The average bubble area of 0.019 mm² at 500 μL/min may be negligible because this is less than 0.5% of the reaction chamber area of 12.57 mm² and only occurred for two instances. The SDO shape had the largest average bubble sizes and highest incidences of bubbles, followed by the SDIO shape. There were no bubbles for any of the flow rates for the SDT shape (represented by dots 86).

The overall percentage of chambers with bubbles for each reaction chamber design is shown in a graph 88 in FIG. 5B, which counts the number of chambers that had bubbles for each shape out of ninety reaction chamber repetitions when flow rate is not considered. Preliminary testing of a circular reaction chamber with no same-depth (NSD) extensions had bubbles form in all the wells, so this shape was not detailed, but its percentage is shown for comparison of the usefulness of a same-depth extension. The percentages of chambers with bubbles were 100%, 17.8%, 7.8%, and 0% for NSD, SDO, SDIO, and SDT, respectively. Only bubbles that formed in the circular well area 26 of the reaction chamber 18 were considered, such as the example in FIG. 5C, which shows two bubbles 89 in the well 26. Sometimes bubbles 89 formed in the same-depth extension 28/30, like that shown in FIG. 5D, but these were not counted because they were immobilized in the same-depth extension and unlikely to interfere with a reading of fluorescence at the center well 26. The bubbles 89 trapped in the same-depth extensions 28/30 may also show how the structures are helping to reduce bubble formation in the analysis area of the reaction chambers 18.

Accordingly, in all three devices 10A, 10B, 10C, adding a same-depth extension to circular wells 26 was found to reduce instances of bubble formation compared to having traditional designs with no same-depth extensions (NSD). In some embodiments, providing same-depth extensions on both the inlet and outlet side of the reaction chamber, like the device 10B having an SDIO design, may be beneficial over providing same-depth extension on only a single side of the reaction chamber 18, like the device 10A having an SDO design, as the SDIO design showed to further reduce bubble formation compared to the SDO design. As noted above, the SDIO design was found to reduce the instances of bubble formation by 92.2% across different flow rates compared to traditional designs. Furthermore, in some embodiments, providing same-depth extensions on both the inlet and outlet side of the reaction chamber, like the device 10B having an SDIO design, may be beneficial over making the entire design have the same depth like the device 10C having an SDT design, despite the SDT design completely eliminating bubble formation, to reduce the cost of reagents and to better separate different primer targets if considering multiplexing. For example, in multiplexed assays where multiple reaction chambers 18 are used to detect different targets simultaneously, the reduced channel volume of the SDIO design may minimize cross-contamination between chambers and reduce the amount of sample and reagents needed to fill the device 10. The smaller channel volumes may also provide better fluidic isolation between different reaction chambers 18, allowing each chamber to function independently for detecting distinct primer targets.

The underlying mechanism for bubble reduction in the SDIO design involves a unique filling pattern that promotes laminar flow. As demonstrated in FIG. 5E, the SDIO shape fills from side-to-side rather than bottom-to-top. Specifically, fluid first fills to the top of the same-depth inlet extension 28 and maintains a steady fluid front through to the other side of the analysis area (e.g., the well 26), then fills the same-depth outlet extension 30 completely. Once the inlet extension 28 is filled, laminar flow commences, allowing fluid to flow evenly through the well 26 to the outlet extension 30. In contrast, for shapes like NSD, fluid fills from bottom to top, which is more conducive to bubble formation because there is no path to remedy air pockets. An example of an NSD shape filling is shown in FIG. 5F, where fluid initially filled the bottom of the well completely and kept going up through the fourth frame, but then an air bubble 89 suddenly formed because the entire height of the fluid cannot touch the top cover at the same time. Air bubbles that form are too big to escape through the thin vent 14 and, therefore, stay in the well 26.

Additionally, as the SDT design was shown to result in no bubbles, an SDIO design (or SDO design) with longer extension(s) 28/30 may be able to reduce bubbles even further. More specifically, in the testing of the SDO and SDIO designs, as described above, flow rate had an effect on the formation of bubbles. As such, for POC applications, flow rate may need to be considered based on the end user, where a metered flow rate may be helpful for all SDO or SDIO designs, or a modification of the design to have longer extension(s) 28/30 may eliminate any flow rate effect to allow for manual processing of fluid samples by hand, eliminating the need for precise metering systems that would complicate POC use.

The bubble reduction achieved by the SDIO and SDO designs may enable the use of quantitative fluorescence-based detection methods. In conventional microfluidic designs where bubbles frequently form, devices may be limited to qualitative colorimetric detection, which provides simple positive or negative results but lacks the sensitivity and dynamic range needed for quantitative analysis. Bubbles interfere with fluorescence measurements by scattering light, creating inconsistent optical paths, and occupying space within the reaction chamber that should contain the assay mixture. By substantially reducing or eliminating bubble formation, the reaction chamber designs described herein may enable reliable quantitative fluorescence measurements, allowing for determination of target concentrations and providing greater analytical sensitivity. This quantitative capability may be particularly valuable in point-of-care applications where accurate measurement of pathogen load or biomarker levels is desired.

### Nuclease Contamination

For the microfluidic devices described herein to be effective in point-of-care settings, they should not only fill without bubbles but also be free from nuclease contamination that could interfere with nucleic acid amplification assays. That is, in both POC and laboratory environments, a possible risk with microfluidics is their susceptibility to nuclease contamination, which can inhibit nucleic acid amplification assays. A set of experiments was performed to check microfluidic fabrication processes for nuclease contamination, and techniques are provided, according to some embodiments, that can help to reduce the contamination.

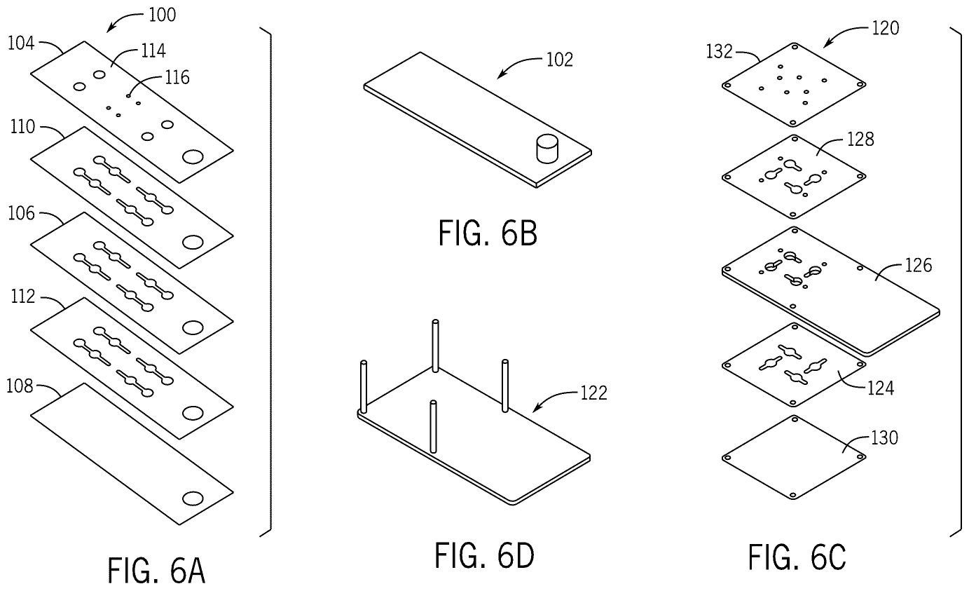

In the experiments, nuclease contaminated microfluidic devices 100 were fabricated using the design shown in FIG. 6A, with an alignment fixture 102 shown in FIG. 6B. The fixture 102 was made from cast acrylic and a 6.35 mm diameter stainless steel dowel pin (McMaster-Carr). The device 100 shown in FIG. 6A simulates low-cost microfluidic devices which are made using only xurography when laser cutters and other equipment are not available. Additionally, the methods of contamination and decontamination take into consideration low-resource settings. The device 100 is 75 mm long, 25 mm wide, and 0.4 mm high. A top cover 104, middle layer 106, and bottom cover 108 are 75 μm Melinex® 454 PET (Tekra) and an upper layer 110 and lower layer 112 are 81 μm double-sided PSA tape (90445Q, Adhesives Research®). All components were cut with Cricut® Explore and touched with bare fingers to simulate contamination. Following contamination, gloves were used for the remaining steps, including the assembly process. All layers 104-112 were wiped with 80% ethanol (Fisher Scientific). To assemble a device 100, the bottom cover 108 was placed on the alignment fixture 102, and then the bottom PSA layer 112 was placed on top. The stacking of the layers from bottom to top continued until the top cover 104 was placed. The completed chip 100 was pressed in a 500-lb capacity arbor press.

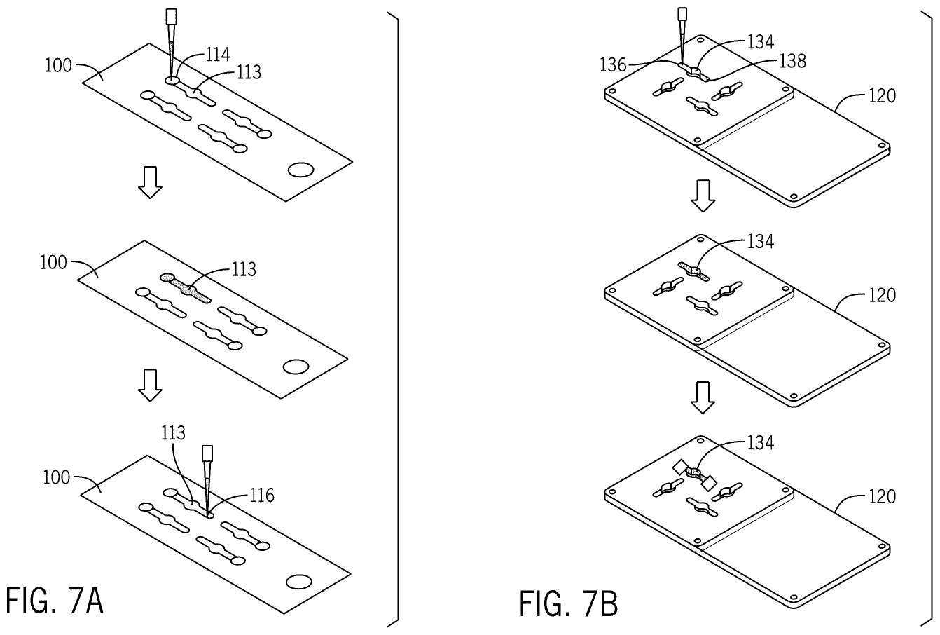

In the experiment, there were five different treatments to test for decontamination on the device 100. The first treatment was when the wiped layers were assembled (ASM). The second treatment was when the wiped layers were assembled, and then the completed chip 100 was placed at the line of sight of an ultraviolet C light (LTC30T8, LightTech) in a biosafety cabinet for 20 minutes on each side (A-UV). The third treatment was when the wiped layers were assembled, a pipette was used to aspirate 80% ethanol through each reaction chamber 113 for a rinse, and the completed chip 100 was placed near UV light for twenty minutes on each side (A-ER-UV). For example, in the top cover 104, there are four larger holes 114 which serve as inlets and four smaller holes 116 which serve as vents for reaction chambers 113 (shown in FIG. 7A). As shown in FIG. 7A, ethanol was placed at the inlet 114 and aspirated out of the vent 116 using a pipette. The fourth treatment was when each layer was placed near UV light for twenty minutes on both sides prior to assembly. The chip 100 was then assembled, and the completed chip 100 was placed near UV light again for twenty minutes on both sides (UV-A-UV). Metal wires and polyvinyl chloride (PVC) pipes were used to hang the material layers and completed chips from the top of a biosafety cabinet (3440009, Labconco) so that they were 19 to 28 cm away from the UV light and directly at the line of sight. Three chips 100 of each treatment were made.

To detect nuclease contamination, RNaseAlert™ and DNaseAlert™ kits from Integrated DNA Technologies (IDT) were used. Samples from the chips 100 were collected by passing 100 μL of nuclease-free water (IDT) through each chip 100 by using a pipette. About 15 μL of water was placed at the inlet 114 of each reaction chamber 113 and aspirated out of the vent 116, like that demonstrated in FIG. 7A. This was repeated until each chamber 113 was collected at least twice and all the water from the same chip 100 was combined. Following established IDT protocols, of the 100 μL of water aspirated through each chip 110, 45 μL of the sample was used for RNaseAlert™ and 40 μL of sample was used for DNaseAlert™. The remaining reagents from each kit were added and both assays were allowed to incubate at 37° C. for 60 minutes. The samples were transferred to a clear-bottom 96-well microplate (Greiner) and analyzed in a SpectraMax® M5 microplate reader. The RNase samples were read at 490-520 nm and the DNase samples were read at 536-556 nm. This was the first collection of samples from each chip 100.

The fifth treatment involved reusing all the nuclease contamination chips 100 and giving them all the same additional treatment for a second collection of samples. For each chip 100, a pipette was used to aspirate 80% ethanol through each of the chambers 113 and each chip 110 was placed near UV light for 20 minutes on each side. Again, 100 μL of nuclease-free water was aspirated through the chambers 113 of each chip 100 and the samples were used with RNaseAlert™ and DNaseAlert™ kits. This second collection of samples was also analyzed in a SpectraMax® M5.

Furthermore, to simulate fabrication environments where a laser cutter is available, a laser cut chip 120, as shown in FIG. 6C, was used with an alignment fixture 122, as shown in FIG. 6D. The chip assembly was similar to the general assembly process described in FIG. 4, where a lower PSA layer 124 is placed on the alignment fixture 122, then an acrylic middle layer 126, and both were removed to continue placement of the next layer (e.g., an upper layer 128, a bottom cover 130, and a top cover 132). The middle layer 126 is 1.5 mm thick black cast acrylic (McMaster-Carr) and the upper and lower layers 124, 128 are 81 μm double-sided PSA tape (90445Q, Adhesives Research®). The acrylic and PSA materials come with protective liners on both sides, so bare fingers were not run across the materials and gloves were used for all processes. The laser cut chip 120 is 70 mm long, 36 mm wide, and 1.9 mm high.

There were three treatments to check for contamination in the laser cut chip design. The first treatment was when the chip 120 was assembled with 75 μm Melinex® 454 PET (Tekra) covers 130, 132 on the top and bottom (ASM). The second treatment was reusing the same chip 120 to do an 80% ethanol rinse and then placing the chip 120 near UV for twenty minutes on each side (A-ER-UV). The third treatment was making a chip 120 with single-sided PSA tape (MTC Bio) as the cover 130, 132 on the top and bottom (PSA). The cast acrylic and PSA tape came with protective liners while the PET did not, so this was to check if nucleases are introduced in a gloved environment. There were six total chips 120 made for these treatments. The acrylic thickness allows for more volume, so 120 μL of nuclease-free water was passed through reaction chambers 134 of each chip 120 (e.g., by placing the sample at an inlet 136, as shown in FIG. 7B). The samples were used with RNaseAlert™ and DNaseAlert™ kits, and the assay was allowed to incubate for 60 minutes then analyzed in a SpectraMax® M5.

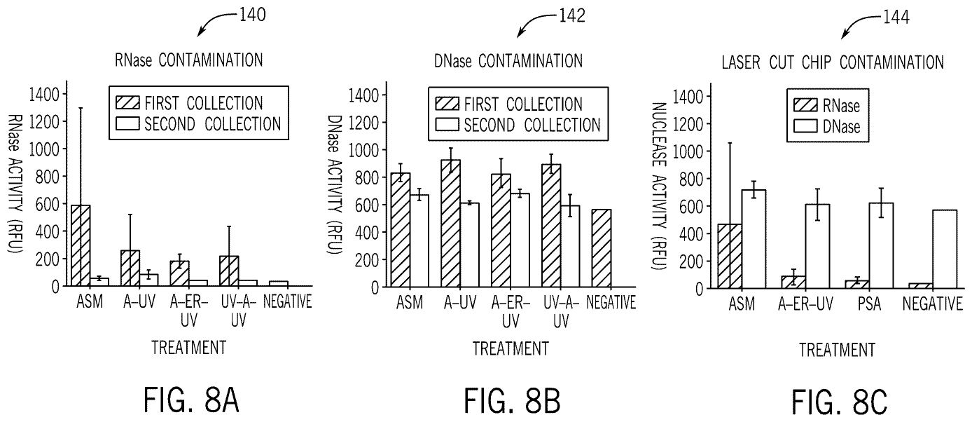

Looking to results of these experiments, the various treatments to the materials or chips 100, 120 are summarized with the following abbreviations: assembled (ASM and A), UV light for twenty minutes on each side (UV), and ethanol rinse (ER). The order of the abbreviations indicates the order of treatments for each set of chips. The results of the nuclease contamination chip 100 from FIG. 6A are illustrated in the graphs 140, 142 shown in FIGS. 8A and 8B, respectively. Experimentation of this chip 100 was to simulate low-cost microfluidic devices made using only xurography, so all layers were touched with bare fingers to mimic heavy contamination.

In the results graph 140 of FIG. 8A, for RNase contamination, for the assembly only treatment (ASM), there was more initial RNase contamination present, indicating that simply wiping the layers with ethanol and assembling may not be enough. The three treatments of A-UV, AER-UV, and UV-A-UV were all similarly able to reduce contamination when compared to the assembly only treatment, shown in the darker shade. From the first collection of samples, the A-ER-UV treatment was more consistently able to reduce contamination as seen by the smaller error bars (note, the error bars represent standard deviation for three representative chip measurements). This is why the second collection of samples, indicated by lighter shade, applied a second ethanol rinse and UV light exposure to all treated chips, which was able to reduce the RNase contamination significantly.

In the results graph 142 of FIG. 8B, for DNase contamination, there was some initially present across all treatments indicated by darker shade, but for the second collection in light shade, applying an ethanol rinse and UV light exposure to all treated chips 100 did show a reduction. The RNaseAlert™ and DNaseAlert™ tests have different relative fluorescence units (RFU) because they use different reporter dyes and are read at different emission wavelengths. Accordingly, to reduce nuclease contamination, rinsing the microfluidic chip with ethanol and applying UV light to both sides may be a helpful method.

The results of the laser cut chip 120 of FIG. 6C are illustrated in the graph 144 shown in FIG. 8C, where a similar effect can be seen. Experimentation of this chip 120 was to simulate cleaner environments where a laser cutter is also available, so the materials were not touched with bare fingers. Additionally, the adhesive and acrylic used to make this chip 120 come with protective liners, so contaminating them would be futile. The PET does not come with protective liners and may have some nuclease contamination, which is seen in the assembly only treatment (ASM). There is a significant reduction in RNase contamination when the laser cut chip made with PET covers has an ethanol rinse and UV light exposure, shown by the A-ER-UV treatment. When using a PSA cover, all materials of the laser cut chip have a protective liner and do not show much initial contamination. For DNase contamination, the laser cut chip may have had a small initial amount for the assembly only treatment, but the overall presence of DNase in each treatment was very low. For reference, the positive control for both RNase and DNase from the kits is nearly 3500 RFU, but is not shown in FIG. 8C to show more distinction between the treatments.

Generally, the effect of nuclease contamination in point-of-care microfluidics has not been widely reported. In laboratory settings, decontamination methods have been reported for surface and reagent contamination, but there is not a single technique that is effective against all types of contaminating sources. Even when preventative practices are used, such as designated nuclease-free laboratory areas, lab coats, and gloves, nuclease contamination can occur over a span of several years. For POC devices to detect trace amounts of target nucleic acids, it would be beneficial for microfluidic components to be nuclease-free during fabrication and at the testing site.

In the decontamination methods described above, according to some embodiments, ethanol was used to clean microfluidic surfaces because it evaporates completely and does not leave a residue. While there are commercially available nuclease decontamination solutions which inactivate nucleases, these can leave a residue that interacts with assays or interferes with the detection method. Additionally, UV light has been found to inactivate RNase and change its conformation. As such, the decontamination methods described herein can use both ethanol and UV light, which showed promising nuclease reduction for microfluidics made with PET covers. In some applications, the number of ethanol rinses and length of UV exposure can be increased and may depend on the amount of contamination present in the fabrication method. For example, when using microfluidic components that come with protective liners, it may not be necessary to decontaminate if they are peeled and used in aseptic environments, and materials such as PSA tape need not be used with ethanol on the adhesive side. The effect of nuclease contamination may ultimately depend on the assay developed and the microfluidic design used and, as such, the level of nuclease contamination may be dependent on chosen materials and fabrication methods as well as what amount detected may be tolerable for the specific assay.

### Assay Validation

Generally, LAMP and PCR reactions run for 30-60 minutes, so the temperature profile of a laser cut chip 120 (e.g., as shown in FIG. 6C) was measured to see if a desired stable temperature can be obtained with a heating apparatus. More specifically, experiments were conducted where the chip design of FIG. 6C was instrumented with type K thermocouples (5SRTC-TT-36-36, Omega) and inserted into two reaction chambers diagonal from each other. Both sides of the chip 120 were covered with single-sided PSA tape (MTC Bio) and the wells of the reaction chambers 134 were filled with water. The chip 120 was placed on a hot plate (HS61, Torrey Pines Scientific) set at 60° C. and the chip 120 was covered with aluminum foil, which allowed the temperature in the wells 134 to reach 62-63° C. (noting that the control temperature and temperature on the surface of the hot plate can differ). The chip temperature was recorded for 30 minutes with a thermocouple data logger (RDXL4SD, Omega) and the average temperature of the two wells was graphed.

Furthermore, in a set of assay validation experiments, five different material assemblies were tested, including four different types of adhesives from different brands, and PET to eliminate effects from adhesive. Three off-the-shelf PCR plate seals, which are all clear single-sided PSA tape, were chosen for chip compatibility testing: Applied Biosystems 4360954, MTC Bio P1001-PCR, and Thermo Scientific® AB0558. The Applied Biosystems and Thermo Scientific® PCR seals have polyester backing, while the MTC Bio seal has polypropylene backing. The fourth type of adhesive was Adhesives Research® 94090, which is made of polypropylene backing and silicone adhesive. The fifth material was 75 μm Melinex® 454 PET (Tekra). The upper and lower layers 124, 128 for all chips were 81 μm double-sided PSA tape (90445Q, Adhesives Research®). Each of the five material treatments was used as the cover layers 130, 132 in FIG. 6C to make two chips 120, for a total of ten chips 120. Pressure was applied manually around the edges of the reaction chambers 134 rather than placing the assembled chips 120 in an arbor press, because of the thin nature of the channels and single-sided PSA tape used as a cover. Each chip 120 of different material was placed in a Lionheart™ FX automated fluorescence microscope to check for autofluorescence of the materials. The autofluorescence of three reaction chambers 134 from each chip 120 was measured.

Material compatibility was further checked with LAMP reagents. For each material cover type, two chips 120 were used; the reaction chambers 134 of one chip 120 were filled with a positive reaction mix, and the second chip 120 was filled with negative reaction mix. The outer four holes 136 near the reaction chambers 134 were for filling with a pipette, and the inner four holes 138 were for venting. Each chamber 134 was filled with about 30 μL of the reaction mix and the holes 136, 138 were sealed with a square of the respective adhesive material, as demonstrated in FIG. 7B. For the PET cover chips 120, the holes 136, 138 were sealed with the PSA tape from Applied Biosystems. The filled chips 120 were placed on a hot plate, covered with aluminum foil, and heated for 30 minutes. The completed reactions were checked in a Lionheart™ FX automated fluorescence microscope. The fluorescence of all reaction chambers 134 of each chip 120 was measured.

The reaction mix followed the WarmStart® LAMP kit protocol from New England BioLabs, which uses a 2× master mix. The main components of the mix were 12.5 μL of 2X master mix, 0.5 μL of 250 mM SYTO 9 green fluorescent nucleic acid stain (Invitrogen, Eugene, OR), 2.5 μL of 10× primer mix, and 4.5 μL of water. For the positive reaction mix, 5 μL of a template was added, and for the negative reaction mix, 5 μL of water was added. The template was heat-inactivated SARS-COV-2 omicron variant (VR3347HK, ATCC) diluted to a stock amount of 10,000 copies per μL, so each reaction chamber had 50,000 copies. The primer mix targeted the open reading frame (ORF) of SARS-COV-2, and the sequences were custom designed and mixed following the WarmStart® LAMP kit primer mix protocol. The positive and negative reaction mixes were verified in a commercial qPCR system (Chai) to ensure amplification for the positive mix and no amplification for the negative mix. The reaction mixes were scaled up to fill all chips 120.

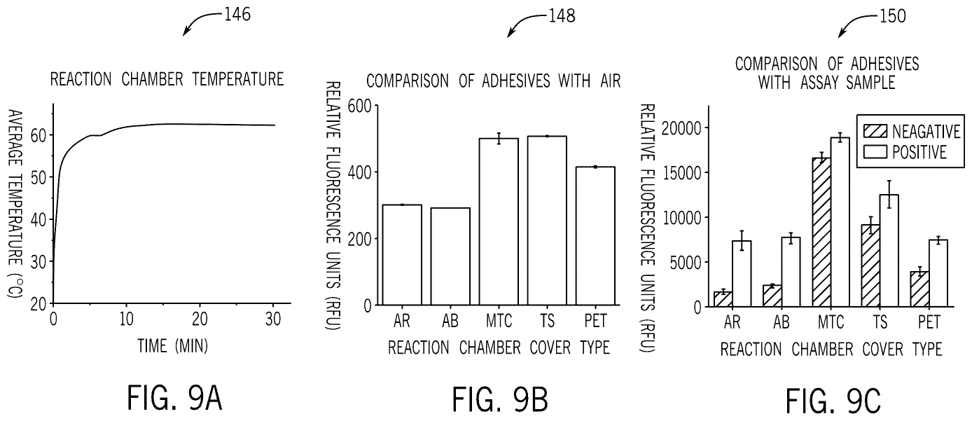

Looking to the results of these experiments, the laser cut chip temperature stability and autofluorescence of materials were checked before assay validation tests. The temperature profile shown in the graph 146 of FIG. 9A shows that the laser cut chip 120 can reach a stable temperature amenable for nucleic acid amplification using the chosen heating apparatus. It took close to ten minutes for the reaction chambers 134 to heat up and reach the appropriate temperature, which held steady indefinitely. For the two reaction chambers 134 measured in this experiment, the average temperature after ten minutes was around 62.6° C. While a hot plate was used for this experiment, the setup shows that a heater can be designed to keep a stable temperature in the reaction chambers 134. The amount of time for a microfluidic chip to reach an assay temperature may depend upon the type of heater used in the POC device and the final microfluidic design. As such, heater designs may need to adjust for the difference in temperature of the heating element and microfluidic chip for more accurate heat transfer.

As noted above, to check the autofluorescence of the materials, the laser cut chip 120 of FIG. 6C was made with five different types of covers 130, 132. Four of the covers were different types of adhesives from different brands. The adhesives are referred to by their brand abbreviation: Adhesives Research® 94090 (AR), Applied Biosystems 4360954 (AB), MTC Bio P1001-PCR (MTC), and Thermo Scientific® AB0558 (TS). The fifth type of material was a plastic cover (PET) to eliminate any interactive effects from adhesive. A graph 148 illustrated in FIG. 9B shows the results from an automated fluorescence microscope measurements of three empty reaction chambers 134 for each chip 120, with error bars representing standard deviation. This graph 148 shows the base fluorescence of each material, with AR and AB having the least autofluorescence, followed by PET, and then MTC and TS with the highest autofluorescence. When choosing microfluidic materials for fluorescence-based assays, materials with low autofluorescence can allow for more discriminability in the results.

Additionally, as noted above, the laser cut chip design of FIG. 6C was validated with a LAMP assay, with the results shown in a graph 150 of FIG. 9C. Each material cover type had two chips 120 made: one for a positive reaction mix and one for a negative reaction mix. The chips 120 were heated for 30 minutes and then the fluorescence of three reaction chambers 134 from each chip 120 were measured on an automated microscope. The error bars on the graph 150 represent standard deviation.

Referring still to FIG. 9C, all positive LAMP reaction mixes in all chips 120 were able to amplify, as shown by the higher fluorescence of each positive chip. The ability to perform quantitative fluorescence measurements demonstrates an advantage of the bubble-reducing reaction chamber designs, as bubble formation in conventional designs would interfere with such measurements and limit devices to qualitative colorimetric detection. There are significant differences between the performance of each material. The fluorescence ratio between the positive to negative chips for each type of material is as follows: 4.63 for AR, 3.36 for AB, 1.14 for MTC, 1.38 for TS, and 1.90 for PET. AR shows the strongest difference between positive and negative reactions, while MTC shows the least. For POC fluorescence detection, having the strongest difference between positive and negative samples can be beneficial as there may be trade-offs in device design that reduce signal intensity detection to allow for mobility or lower device costs. While all adhesive materials were labeled as nuclease-free or PCR-inhibitor free, it was found that some may be better than others to use for microfluidic chip fabrication. Material compatibility with PCR has been reported before, where different materials were broken into small pieces and then placed into reaction tubes for thermal cycling. In contrast, the experiments performed here allowed for more realistic LAMP assay performance of different materials because they were heated with reagents in reaction chamber designs that are likely similar in surface area and volume to the final product. Additionally, the methods described here can be expanded to PCR.

In view of the above, in an example commercial embodiment, a point-of-care diagnostic device may comprise a disposable microfluidic chip as described herein, pre-loaded with dried LAMP reagents in the reaction chambers 18. A user may add a patient sample (such as a nasal swab in buffer) to the device inlet 12 using a provided transfer pipette, manually inject the sample through the device, place the device in a portable heating block, and read results using a smartphone-based fluorescence reader after 30 minutes. The bubble-free filling enabled by the reaction chamber designs described herein can ensure reliable quantitative results without requiring trained laboratory personnel or expensive equipment.

In light of the above, some embodiments provide several bubble reduction, fabrication, decontamination, and material selection methods to increase the success of microfluidic devices for nucleic acid detection in POC or low-resource settings. Air entrapment in microfluidics used for liquid assays is a concern because bubbles may affect signal detection. To mitigate this common issue, some embodiments provide designs to reduce bubble formation, such as the SDIO (as well as the SDO and SDT) designs. That is, bubble formation can be reduced by having same-depth extensions on the inlets and/or outlets of reaction chambers, with the co-planar configuration enabling side-to-side filling and promoting laminar flow through the reaction chambers. Increasing the length of the extensions may increase success of the reaction chamber to fill completely without bubbles. The reaction chamber designs are amenable to pressure-driven flow and can accommodate varying human-applied forces, eliminating the need for auxiliary mechanical or pneumatic actuators to regulate flow rate. This allows the devices to be operated with simple manual actuation, such as hand-operated syringes, without requiring precise metering systems that would complicate POC use.

The complete filling reproducibility of the reaction chamber designs enables quantitative fluorescence-based detection, rather than being limited to qualitative colorimetric detection as with conventional bubble-prone designs. This quantitative capability provides greater sensitivity and allows for measurement of target concentrations. Additionally, fabricating microfluidic chips using xurography and/or laser cutting techniques can keep costs low and are easier for unskilled personnel to assemble through lamination. For example, alignment fixtures that have posts to match up to alignment holes in each material layer can allow for accurate alignment of each layer and their shape cutouts. Thus, using an alignment fixture can be a cheaper way for personnel to accurately assemble in low-resource settings and requires little prior knowledge. The reaction chamber designs may be integrated into any microfluidic chip layout to accommodate existing or new device configurations, and may be configured for multiplexed detection where multiple reaction chambers are used to simultaneously detect different targets.

Additionally, as nuclease contamination is not widely reported for microfluidics, methods are provided herein, according to some embodiments, to reduce contamination by using a combination of ethanol rinses and UV light exposure, which may reduce contamination up to tenfold and, in some instances, may be more helpful for microfluidics made with plastic film covers. For microfluidics using materials that come with protective liners, decontamination may not be necessary if they are opened and used in aseptic environments. The methods described herein were further validated by running successful LAMP assays, and these methods can be expanded to PCR. The commercial potential of these innovations is particularly beneficial for point-of-care applications, where untrained users in low-resource settings require simple, reliable, and cost-effective diagnostic solutions that can be deployed without sophisticated laboratory infrastructure or extensive technical training.

As used herein, the terms “microfluidic device” and “chip” may be used interchangeably. The term “reaction chamber” refers to the entire chamber structure including the well and extensions, while “well” refers specifically to the circular central portion. The terms “same-depth extension” and “co-planar extension” refer to extensions that are at the same depth as the reaction chamber well.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. A microfluidic device comprising:
a top surface including an inlet and a vent extending therethrough; and
a layer positioned beneath the top surface, the layer comprising:
a reaction chamber,
a first channel in fluid communication between the inlet and the reaction chamber,
an inlet extension between the first channel and the reaction chamber, and
an outlet extension between the reaction chamber and the vent,
wherein the reaction chamber, the inlet extension, and the outlet extension have an equal depth and are co-planar.

2. The microfluidic device of claim 1, wherein the reaction chamber, the inlet extension, and the outlet extension have a first depth, and the first channel has a second depth less than the first depth.

3. The microfluidic device of claim 1, wherein the first channel has the equal first depth.

4. The microfluidic device of claim 1, wherein the top surface is a top cover disposed over the top layer.

5. The microfluidic device of claim 1, wherein the layer further comprises a second channel in fluid communication between the reaction chamber and the vent, wherein the outlet extension is positioned between the reaction chamber and the second channel.

6. The microfluidic device of claim 1, wherein the layer includes a first layer, a second layer, and a third layer, wherein the first channel extends through third layer and the reaction chamber extends through the first layer, the second layer, and the third layer.

7. The microfluidic device of claim 6, further comprising a bottom cover positioned beneath the third layer.

8. The microfluidic device of claim 1, wherein the inlet extension has a length between 2 mm and 5 mm.

9. The microfluidic device of claim 1, wherein the reaction chamber has a diameter between 2 mm and 10 mm.

10. The microfluidic device of claim 1, further comprising a hydrophobic membrane filter positioned over the vent.

11. The microfluidic device of claim 1, wherein the layer comprises multiple reaction chambers, each reaction chamber having a respective inlet extension and outlet extension with the equal depth.

12. The microfluidic device of claim 1, wherein the reaction chamber is circular in shape.

13. A method of fabricating a microfluidic device, the method comprising:
cutting, through a top cover with a cutting plotter, an inlet hole, a vent hole, and a first alignment hole;
cutting, through a layer with the cutting plotter, a second alignment hole, a first channel, a reaction chamber, an inlet extension extending from the reaction chamber, and an outlet extension extending from the reaction chamber, wherein the reaction chamber, the inlet extension, and the outlet extension have an equal depth;
placing the top cover over an alignment fixture by aligning the first alignment hole of the top cover with a post of the alignment fixture; and
placing the layer over the top cover by aligning the second alignment hole of the layer with the post of the alignment fixture to adhere together the top cover and the layer so that the inlet hole is in fluid communication with the first channel and the vent hole is in fluid communication with the outlet extension.

14. The method of claim 13, wherein the inlet extension and the outlet extension each have a length between 2 mm and 5 mm.

15. The method of claim 13, further comprising cutting the layer with a laser cutter when the layer comprises acrylic material.

16. The method of claim 13, further comprising pressing the assembled microfluidic device in a press to ensure adhesion between layers.

17. The method of claim 13, further comprising placing a hydrophobic membrane filter over the vent hole.

18. The method of claim 13, further comprising treating the microfluidic device with ethanol and UV light exposure.

19. A method of performing a nucleic acid amplification assay, the method comprising:
manually injecting a fluid sample into an inlet of a microfluidic device, the microfluidic device comprising a reaction chamber with an inlet extension and an outlet extension that are co-planar with the reaction chamber;
filling the reaction chamber with the fluid sample in a side-to-side manner through the inlet extension and the reaction chamber to the outlet extension;
heating the microfluidic device to perform nucleic acid amplification of a target in the fluid sample within the reaction chamber; and
detecting the target using quantitative fluorescence measurement of the reaction chamber.

20. The method of claim 19, wherein the nucleic acid amplification assay is a loop-mediated isothermal amplification (LAMP) assay performed at a temperature between 60° C. and 65° C.
